(19) United States
(12) Patent Application Publication
(10) Pub. No.: US 2025/0208241 A1
(43) Pub. Date: Jun. 26, 2025

(54) RECEIVE COIL UNIT, MEDICAL IMAGE DIAGNOSTIC SYSTEM, AND COIL COVER

(71) Applicant: FUJIFILM Corporation, Tokyo (JP)

(72) Inventors: Yosuke OTAKE, Tokyo (JP); Takeshi TANIGUCHI, Tokyo (JP); Koichi ARAI, Tokyo (JP); Kojiro IWASAWA, Tokyo (JP)

(73) Assignee: FUJIFILM Corporation, Tokyo (JP)

(21) Appl. No.: 19/000,717

(22) Filed: Dec. 24, 2024

(30) Foreign Application Priority Data
Dec. 26, 2023 (JP) 2023-220063

(51) Int. Cl. G01R 33/34; A61B 5/055; G01R 33/341

(52) U.S. Cl. CPC G01R 33/34015; A61B 5/055; G01R 33/341

(57) ABSTRACT

Provided are a receive coil unit, a medical image diagnostic system, and a coil cover with which heat can be released from between the receive coil unit and a subject to an outside. A receive coil unit includes: a coil element that receives a nuclear magnetic resonance signal of a subject; a flexible coil cover that covers a periphery of the coil element; and a plurality of passage forming members disposed on an outer side surface of the coil cover that comes into contact with the subject, the plurality of passage forming members forming a first passage extending in a first direction and a second passage extending in a second direction different from the first direction between the subject and the coil cover.

### CROSS-REFERENCE TO RELATED APPLICATION

The present application claims priority under 35 U.S.C § 119 (a) to Japanese Patent Application No. 2023-220063 filed on Dec. 26, 2023, which is hereby expressly incorporated by reference, in its entirety, into the present application.

### BACKGROUND OF THE INVENTION

### 1. Field of the Invention

The present invention relates to a receive coil unit, a medical image diagnostic system, and a coil cover.

### 2. Description of the Related Art

In a case in which a subject is imaged by a magnetic resonance imaging (MRI) apparatus, the subject is disposed in an imaging space of a gantry together with a table of a bed apparatus. In this case, a receive coil unit such as a radio frequency (RF) coil that receives a nuclear magnetic resonance (NMR) signal is used in order to obtain an image of an imaging part (for example, a chest or an abdomen) of the subject.

For example, JP2014-094035A discloses an RF coil unit for children. The RF coil unit comprises a support portion that supports a back surface of the subject and that is provided with a coil element, and a surface of the support portion that comes into contact with the back surface of the subject is formed of a cushion material having unevenness.

JP1998-155764A (JP-H10-155764A) discloses an RF coil set comprising an RF coil and a buffer member that relieves contact of the RF coil.

JP2012-130701A discloses a coil in which a plurality of receiver coils are arranged on a flexible blanket.

### SUMMARY OF THE INVENTION

Incidentally, the receive coil unit is mounted in close contact with the subject in order to obtain high sensitivity. Therefore, it is difficult to release heat from the subject, resulting in a structure in which the heat is easily trapped.

In the RF coil unit disclosed in JP2014-094035A, since the cushion material and the subject are continuously in close contact with each other in a plane direction, it is difficult to release heat.

In the RF coil disclosed in JP1998-155764A (JP-H10-155764A), since the buffer member having a sheet shape is interposed between the subject and the RF coil, it is difficult to release heat.

Since the coil disclosed in JP2012-130701A is formed of the blanket, the coil is in close contact with the subject due to the flexibility of the blanket and traps heat, thereby making it difficult to release the heat.

The present invention has been made in view of such circumstances, and an object of the present invention is to provide a receive coil unit, a medical image diagnostic system, and a coil cover with which heat can be released from between the subject and the receive coil unit to an outside.

A first aspect provides a receive coil unit comprising: a coil element that receives a nuclear magnetic resonance signal of a subject; a flexible coil cover that covers a periphery of the coil element; and a plurality of passage forming members disposed on an outer side surface of the coil cover that comes into contact with the subject, the plurality of passage forming members forming a first passage extending in a first direction and a second passage extending in a second direction different from the first direction between the subject and the coil cover.

A second aspect provides the receive coil unit according to the first aspect, in which the passage forming member is formed of one material or a combination of two or more materials selected from the group consisting of a non-magnetic metal, a resin, a closed-cell material, and rubber.

A third aspect provides the receive coil unit according to the first or second aspect, in which the passage forming member has a plate-like shape.

A fourth aspect provides the receive coil unit according to any one of the first to third aspects, in which the passage forming member is configured to be deformable.

A fifth aspect provides the receive coil unit according to any one of the first to fourth aspects, in which the passage forming member is formed of two or more types of members having different shapes.

A sixth aspect provides the receive coil unit according to any one of the first to fifth aspects, in which the passage forming member has a shape in which a groove is formed on a subject side.

A seventh aspect provides the receive coil unit according to any one of the first to sixth aspects, in which the passage forming member has a hollow structure.

An eighth aspect provides the receive coil unit according to any one of the first to seventh aspects, which further comprises: a heat generation source connected to the coil element, in which the passage forming member is disposed at a position corresponding to the heat generation source.

A ninth aspect provides the receive coil unit according to any one of the first to seventh aspects, which further comprises: a heat generation source connected to each of a plurality of the coil elements, in which the passage forming member is disposed at a position corresponding to a plurality of the heat generation sources.

A tenth aspect provides the receive coil unit according to the eighth or ninth aspect, in which the passage forming member is formed of a heat insulating member.

An eleventh aspect provides the receive coil unit according to the fourth aspect, which further comprises: a spacer provided between the passage forming member and the outer side surface of the coil cover.

A twelfth aspect provides the receive coil unit according to any one of the first to eleventh aspects, in which the plurality of passage forming members are arranged in a lattice form.

A thirteenth aspect provides the receive coil unit according to any one of the first to twelfth aspects, in which the coil cover has a bag shape.

A fourteenth aspect provides a medical image diagnostic system comprising: the receive coil unit according to any one of the first to thirteenth aspects; and a magnetic resonance imaging apparatus that processes the nuclear magnetic resonance signal received by the receive coil unit.

A fifteenth aspect provides a coil cover that covers a periphery of a coil element receiving a nuclear magnetic resonance signal of a subject, the coil cover comprising: a plurality of passage forming members disposed on an outer side surface of the coil cover that comes into contact with the subject, the plurality of passage forming members forming a first passage extending in a first direction and a second passage extending in a second direction different from the first direction between the subject and the coil cover.

According to the present invention, it is possible to release heat from between the subject and the receive coil unit to the outside.

### BRIEF DESCRIPTION OF THE DRAWINGS

FIG. 1 is an external perspective view of a medical image diagnostic system according to an embodiment.

FIG. 2 is a schematic diagram showing an internal configuration of an MRI apparatus.

FIG. 3 is an exploded perspective view schematically showing a configuration of a receive coil unit.

FIGS. 4A and 4B are views of the receive coil unit as seen from a noncontact outer side surface and a contact outer side surface.

FIG. 5 is a diagram showing a state in which the receive coil unit is mounted on a subject.

FIG. 6 is a cross-sectional view of the receive coil unit.

FIGS. 7A and 7B are diagrams for describing a usage form of the receive coil unit.

FIG. 8 is a diagram showing a state in which the receive coil unit is mounted on the subject in a direction different from that in FIG. 5.

FIGS. 9A and 9B are diagrams for describing an embodiment of the receive coil unit.

FIG. 10 is a diagram for describing another embodiment of the receive coil unit.

FIGS. 11A and 11B are diagrams for describing still another embodiment of the receive coil unit.

FIG. 12 is a diagram for describing still another embodiment of the receive coil unit.

FIG. 13 is a diagram for describing still another embodiment of the receive coil unit.

FIG. 14 is a diagram for describing still another embodiment of the receive coil unit.

FIG. 15 is a diagram showing an example in which arrangement of signal processing circuits is different from that in FIGS. 4A and 4B.

FIGS. 16A and 16B are diagrams showing arrangement of passage forming members with respect to the receive coil unit shown in FIG. 15.

FIGS. 17A and 17B are diagrams showing arrangement of passage forming members different from that in FIGS. 16A and 16B.

FIG. 18 is a diagram showing arrangement of passage forming members different from a lattice form in the receive coil unit.

### DESCRIPTION OF THE PREFERRED EMBODIMENTS

Hereinafter, a preferred embodiment of the present invention will be described in detail with reference to the accompanying drawings. In the following description and the accompanying drawings, the same constituent elements are denoted by the same reference numerals, and the duplicated description thereof is omitted. In addition, in the following embodiment, in a case in which a plurality of constituent elements are described and listed, it can be interpreted that at least one of the plurality of constituent elements is included.

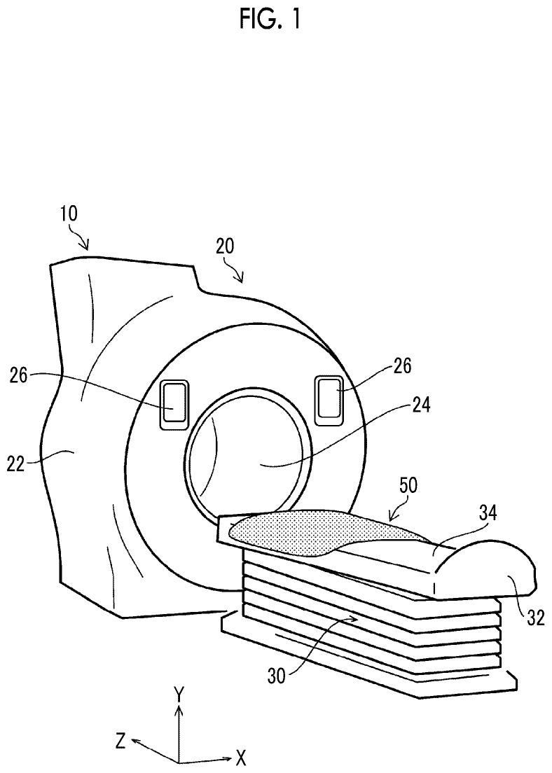

As shown in FIG. 1, a medical image diagnostic system 10 of the embodiment is a magnetic resonance imaging apparatus (hereinafter, referred to as an MRI apparatus) 20, and comprises a receive coil unit 50.

### MRI Apparatus

The MRI apparatus 20 is installed in an examination room of an image diagnostic facility. In the examination room, a subject is placed on a top plate 34 of a table 32 of a bed apparatus 30, and then is transported toward a static magnetic field generating magnet 102 of the MRI apparatus 20 by movement of the table 32.

The MRI apparatus 20 includes the static magnetic field generating magnet 102. The static magnetic field generating magnet 102 has a cylindrical shape and has an imaging space 24 at the center of the cylindrical shape, and the table 32 is moved into the imaging space 24. Gantry monitors 26 are installed on both left and right sides of the static magnetic field generating magnet 102. The gantry monitor 26 also functions as an operation panel.

A three-dimensional coordinate system illustrated in FIG. 1 shows an example of definition of directions in the MRI apparatus 20. An X axis, a Y axis, and a Z axis of the three-dimensional coordinate system are merely examples and the present invention is not limited thereto. For ease of understanding in the following description, the X axis, the Y axis, and the Z axis in the MRI apparatus 20 are defined to be in the same direction in all the drawings. In addition, as an example of the three-dimensional coordinate system, the Z axis direction is a static magnetic field direction, the Y axis direction is an up-down direction of the subject, and the Y axis direction is the same vertical direction as a direction of gravitational force. The X axis direction is a left-right direction of the subject and is a horizontal direction.

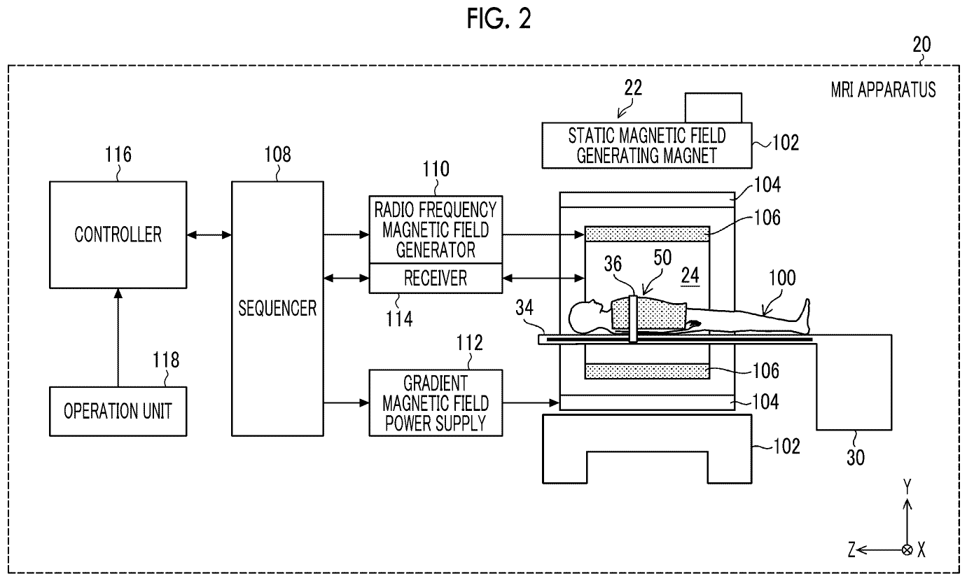

FIG. 2 is a schematic diagram showing an internal configuration of the MRI apparatus 20.

As shown in FIGS. 1 and 2, the MRI apparatus 20 comprises the static magnetic field generating magnet 102, a gradient magnetic field coil 104, and a transmission coil 106.

The static magnetic field generating magnet 102 generates a uniform static magnetic field in the imaging space 24 in which a subject 100 is disposed. The gradient magnetic field coil 104 generates a gradient magnetic field in the imaging space 24. The transmission coil 106 generates, in the imaging space 24, a radio frequency magnetic field for generating a nuclear magnetic resonance (NMR) signal (hereinafter, referred to as an NMR signal) in a nucleus of an atom constituting a tissue of the subject 100.

The receive coil unit 50 is mounted on a chest and an abdomen of the subject 100 placed on the top plate 34 by using a belt 36 provided on the bed apparatus 30. The table 32 on which the subject 100 is placed is moved into the imaging space 24, so that an examination part (imaging target part) of the subject 100 is positioned at the center of the static magnetic field of the imaging space 24. The receive coil unit 50 is mounted on the subject 100 and detects the NMR signal generated from the subject 100.

A sequencer 108 sends commands to a radio frequency transmitter 110 and a gradient magnetic field power supply 112 in accordance with an imaging sequence (pulse sequence), and appropriately amplified signals are sent to the transmission coil 106 or the gradient magnetic field coil 104.

The signal sent to the transmission coil 106 is applied to the subject 100 as a pulsed radio frequency magnetic field (RF pulse) via the transmission coil 106. The NMR signal generated from the subject 100 is detected by the coil element 52 of the receive coil unit 50, and is demodulated by a receiver 114.

The gradient magnetic field coil 104 is composed of gradient magnetic field coils in three directions of X, Y, and Z, and generates gradient magnetic fields according to the signals from the gradient magnetic field power supply 112.

A nuclear magnetic resonance frequency (demodulation reference frequency f0) to be used as a reference for the demodulation in the receiver 114 is set by the sequencer 108. The sequencer 108 performs control such that each unit operates at pre-programmed timing and intensity. Among programs, a program that particularly describes the timing and intensity of RF pulses, gradient magnetic fields, and signal reception is referred to as a pulse sequence.

Various pulse sequences depending on the purpose are known, but the detailed description thereof will be omitted here.

A controller 116 controls an operation of the MRI apparatus 20 via the sequencer 108, and receives the signal demodulated by the receiver 114 and performs various types of signal processing such as image reconstruction. The receiver 114 performs quadrature phase demodulation on the reception signal (NMR signal), which is an analog wave, using the set demodulation reference frequency f0, performs analog-to-digital (AD) conversion, and then transmits the signal to the controller 116. This data is also referred to as a reception signal or measurement data.

The controller 116 receives various instruction inputs from an operation unit 118 and integrally controls respective units of the MRI apparatus 20. In addition, the controller 116 performs processing of performing inverse Fourier transform on the reception signal in a spatial frequency region received via the sequencer 108 to convert the reception signal into an image in a real space, and generates an MRI image.

The controller 116 is implemented by a general-purpose computer such as a personal computer or a microcomputer. The controller 116 comprises a central processing unit (CPU), a read only memory (ROM), a random access memory (RAM), an input/output interface, and the like.

In the controller 116, various programs such as a control program stored in the ROM are loaded onto the RAM, and the programs loaded onto the RAM are executed by the CPU. As a result, the functions of the respective units of the MRI apparatus 20 are implemented, and various types of arithmetic processing and control processing are executed via the input/output interface.

The operation unit 118 includes a mouse, a keyboard, the gantry monitor 26, and the like, and the gantry monitor 26 functions as a part of a graphical user interface (GUI) that receives an input from an imaging staff.

The imaging staff inputs start and stop (including pause) of the MRI apparatus 20, selection of the pulse sequence, an imaging condition, a processing condition, and the like via the operation unit 118.

### Receive Coil Unit of Embodiment

The receive coil unit 50 has a blanket shape, and has a thinner, lighter, and more flexible structure than an integrally molded type. The receive coil unit 50 is deformable to fit the physique of the subject 100, and can image various examination parts. Note that the receive coil unit 50 is an example of a receive coil unit according to the embodiment of the present invention.

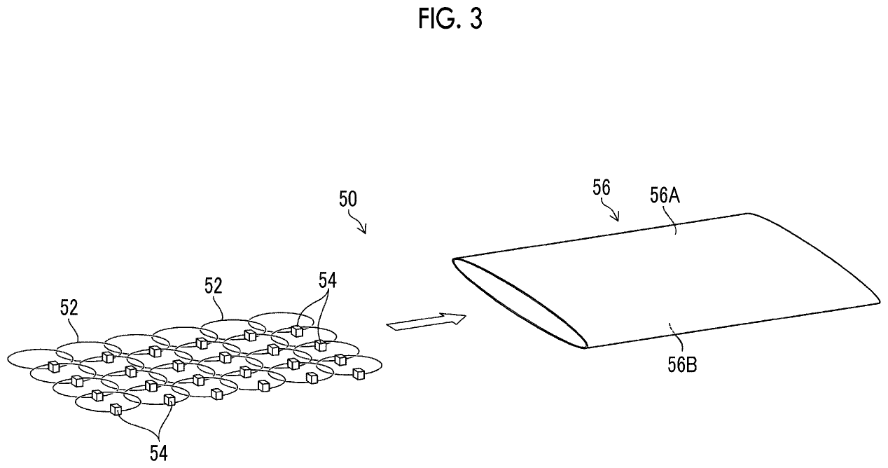

FIG. 3 is an exploded perspective view schematically showing a configuration of the receive coil unit 50. As shown in FIG. 3, the receive coil unit 50 comprises a plurality of coil elements 52, a plurality of signal processing circuits 54 that are electrical components respectively connected to the plurality of coil elements 52, and a coil cover 56.

The coil element 52 functions as a detector (sensor) that receives the NMR signal. The coil element 52 has, for example, a ring-like shape having a diameter of about 10 cm to 15 cm. The coil elements 52 are two-dimensionally arranged inside the coil cover 56. The receive coil unit 50 of this example comprises 24 coil elements 52, making it multi-channel. The number and arrangement of the coil elements 52 are not limited to the example of FIG. 3. The coil element 52 may be configured to be deformable to fit the physique of the subject 100. The coil cover 56 is an example of a coil cover according to the embodiment of the present invention.

The signal processing circuit 54 may be configured as, for example, a module product in which an electric circuit including a plurality of circuit elements is packaged in a cubic or rectangular parallelepiped housing. The signal processing circuit 54 comprises a magnetic coupling prevention circuit for preventing intrusion of the energy emitted from the transmission coil 106 and for removing the coupling between the coil element 52 and the transmission coil 106. The magnetic coupling prevention circuit is configured of a capacitor, a diode, and an inductor. The inductor and the diode are connected in series to form a series circuit. This series circuit is connected in parallel with the capacitor. The diode is connected to a magnetic coupling prevention circuit drive unit. In a parallel resonance circuit consisting of a capacitor, an inductor, and a diode, in a case in which the diode is ON, a resonance frequency can be matched with a resonance frequency of the transmission coil 106, which is adjusted to the same frequency as the magnetic resonance frequency. As a result, the magnetic coupling between the transmission coil 106 and the receive coil unit 50 is prevented. In a case of preventing the magnetic coupling, a part of the energy emitted from the transmission coil 106 is converted into heat in the magnetic coupling prevention circuit. The coil element 52 is an example of a coil element according to the embodiment of the present invention. The signal processing circuit 54 is an example of a heat generation source according to the embodiment of the present invention.

The coil cover 56 is a cover that constitutes a housing body that covers a periphery of the plurality of coil elements 52 and the plurality of signal processing circuits 54 that are heat generation sources. Electrical components including the plurality of coil elements 52 and the plurality of signal processing circuits 54 are housed inside the coil cover 56 to form a blanket-shaped receive coil unit 50. The plurality of coil elements 52 and the plurality of signal processing circuits 54 may be housed inside the coil cover 56 in a state of being fixed on a film (not shown). The film is a support member that fixes a positional relationship between the coil element 52 and the signal processing circuit 54 and suppresses positional deviation.

The coil cover 56 is formed in a bag shape by sewing or gluing end parts of a sheet-shaped material cut into one piece. In this example, a first sheet body 56A and a second sheet body 56B are sewn or glued together to form a bag-shaped coil cover 56. The material of the coil cover 56 may be a urethane-based resin such as polyurethane, a polyamide synthetic resin such as nylon, or the like. A surface of the first sheet body 56A exposed to the outside constitutes an outer side surface that is an outer surface that does not come into contact with the subject (hereinafter, also referred to as a noncontact outer side surface). A surface of the second sheet body 56B exposed to the outside constitutes an outer side surface that is an outer surface that comes into contact with the subject (hereinafter, also referred to as a contact outer side surface). A cable unit (not shown) is connected to the coil cover 56. The cable unit is a unit in which multi-channel cables for obtaining signals from each of the plurality of coil elements 52 are bundled together, and is electrically connected to the plurality of signal processing circuits 54.

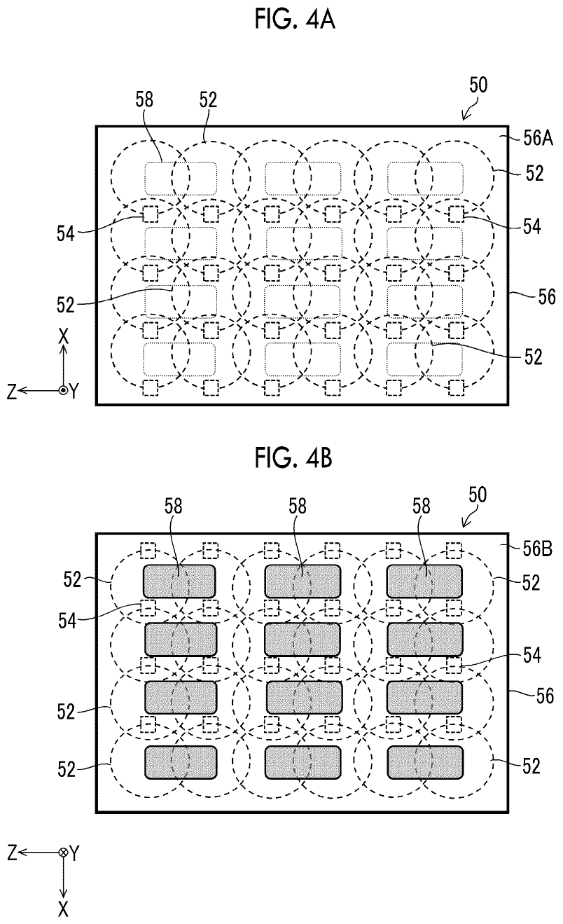

FIGS. 4A and 4B are views of the receive coil unit 50 as seen from the noncontact outer side surface and the contact outer side surface. FIG. 4A is a view as seen from the first sheet body 56A (noncontact outer side surface). As shown in FIG. 4A, the receive coil unit 50 of this example has a substantially rectangular shape in which a side along the Z axis direction is longer than a side along the X axis direction. The shape of the receive coil unit 50 is decided by the shape of the coil cover 56. The shape and size of the coil cover 56 are not particularly limited and can be appropriately changed depending on the imaging part. The coil cover 56 may have, for example, a square shape as seen from the first sheet body 56A. In addition, the flexibility of the receive coil unit 50 may be mainly decided by the characteristics of the coil cover 56. The 24 coil elements 52 housed in the coil cover 56 are regularly arranged two-dimensionally and are disposed over the entire surface of the coil cover 56. Specifically, six coil elements 52 are arranged along the Z axis direction. In addition, four coil elements 52 are arranged in the X axis direction. In addition, the plurality of heat generation sources (signal processing circuits 54) are regularly arranged two-dimensionally in the same manner as the plurality of coil elements 52. The arrangement of the plurality of heat generation sources (signal processing circuits 54) is not limited to an arrangement pattern in which the heat generation sources are regularly arranged two-dimensionally, and may be an arrangement pattern having no regularity.

FIG. 4B is a view as seen from the second sheet body 56B (contact outer side surface). As shown in FIG. 4B, the receive coil unit 50 includes a plurality of passage forming members 58 on the contact outer side surface of the second sheet body 56B. The plurality of passage forming members 58 are regularly arranged two-dimensionally and are arranged in a lattice form. In this example, 12 passage forming members 58 are disposed on the contact outer side surface of the second sheet body 56B. The passage forming member 58 has a substantially rectangular shape in which a side along the Z axis direction is longer than a side along the X axis direction. The passage forming member 58 protrudes from the second sheet body 56B toward the subject 100.

Three passage forming members 58 are arranged along the Z axis direction such that each of the passage forming members 58 straddles two adjacent coil elements 52. In addition, four passage forming members 58 are arranged at positions corresponding to the coil elements 52 along the X axis direction. The shape, the number, and the arrangement of the passage forming members 58 are not limited to the example shown in FIGS. 4A and 4B as long as a passage, which will be described below, is formed. In FIGS. 4A and 4B, the shape of the passage forming member 58 is shown in a different color from the coil cover 56 in order to facilitate understanding of the shape of the passage forming member 58. The coil cover 56 and the passage forming member 58 may be the same color or different colors. The passage forming member 58 is an example of a passage forming member according to the embodiment of the present invention.

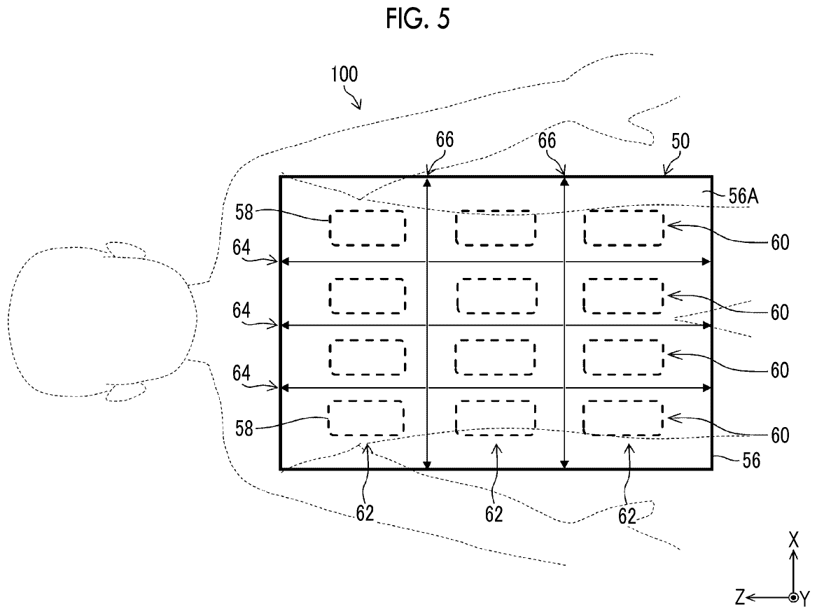

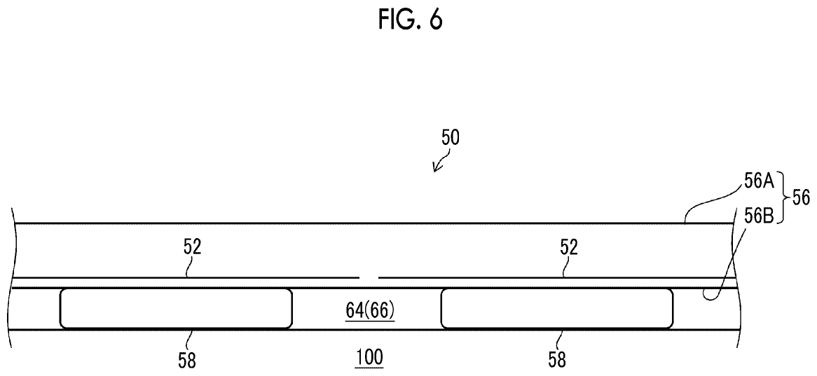

FIG. 5 is a diagram showing a state in which the receive coil unit 50 is mounted on the subject 100. FIG. 6 is a cross-sectional view of the receive coil unit 50.

As shown in FIG. 5, the receive coil unit 50 is configured of the plurality of passage forming members 58 forming four first direction arrangement groups 60 and three second direction arrangement groups 62. One first direction arrangement group 60 is configured of three passage forming members 58 arranged in the Z axis direction. The four first direction arrangement groups 60 are arranged in the X axis direction. One second direction arrangement group 62 is configured of four passage forming members 58 arranged in the X axis direction. The three second direction arrangement groups 62 are arranged in the Z axis direction. The first direction arrangement groups 60 and the second direction arrangement groups 62 have different arrangement directions of the passage forming members 58.

The first direction arrangement groups 60 adjacent to each other form a first passage 64 extending in a first direction (Z axis direction). In this example, three first passages 64 are formed. In addition, the second direction arrangement groups 62 adjacent to each other form two second passages 66 extending in a second direction (X axis direction) different from the first direction. The first passage 64 extends in the Z axis direction over the entire contact outer side surface of the receive coil unit 50. The second passage 66 extends in the X axis direction over the entire contact outer side surface of the receive coil unit 50. In this example, a passage extending along a longitudinal direction of the receive coil unit 50 is referred to as the first passage 64, and a passage in a direction different from the first passage 64 is referred to as the second passage 66. Note that a passage extending along a lateral direction of the receive coil unit 50 may be referred to as the first passage 64, and a passage intersecting the first passage 64 may be referred to as the second passage 66.

The first passage 64 is an example of a first passage according to the embodiment of the present invention. The second passage 66 is an example of a second passage of the present invention.

As shown in FIG. 6, the passage forming members 58 separate the receive coil unit 50 (second sheet body 56B) and the subject 100. In a case in which the passage forming members 58 come into contact with the subject 100, the first passage 64 is formed by the adjacent passage forming members 58, the second sheet body 56B, and the subject 100. The first passage 64 serves as a passage for air. Similarly, the second passage 66 is formed by the adjacent passage forming members 58, the second sheet body 56B, and the subject 100. The second passage 66 serves as a passage for air. The first passage 64 and the second passage 66 promote a flow of air and can release heat through the flow of air. The cross-sectional view of FIG. 6 is schematically described both in a case of being seen from the Z axis direction and in a case of being seen from the X axis direction. The same applies to the following cross-sectional views.

In this example, the passage forming member 58 has a plate-like shape, and a thickness thereof can be appropriately designed. In a case in which the coil element 52 is too far away from the subject 100 in a state in which the receive coil unit 50 is mounted on the subject 100, the sensitivity of signal detection is lowered, so that it is preferable that the passage forming member 58 is not made to have a structure that is thicker than necessary. From this viewpoint, the thickness of the passage forming member 58 is preferably 10 mm or less.

The passage forming member 58 can be formed of one material selected from the group consisting of a non-magnetic metal, a resin, a closed-cell material, and rubber. As the non-magnetic metal, copper, titanium, aluminum, brass, and the like can be applied to the passage forming member 58. As the resin, polycarbonate, a fluororesin, acrylic, and the like can be applied to the passage forming member 58. As the closed-cell material, a bubble cushioning material, polystyrene foam, urethane foam, or the like can be applied to the passage forming member 58. The bubble cushioning material may be formed of a material containing polyethylene. As the rubber, urethane rubber, fluorinated rubber, and the like can be applied to the passage forming member 58. It is preferable that the passage forming member 58 has load-bearing properties sufficient to ensure the first passage 64 and the second passage 66 even in a case in which the weight of the receive coil unit 50 is applied.

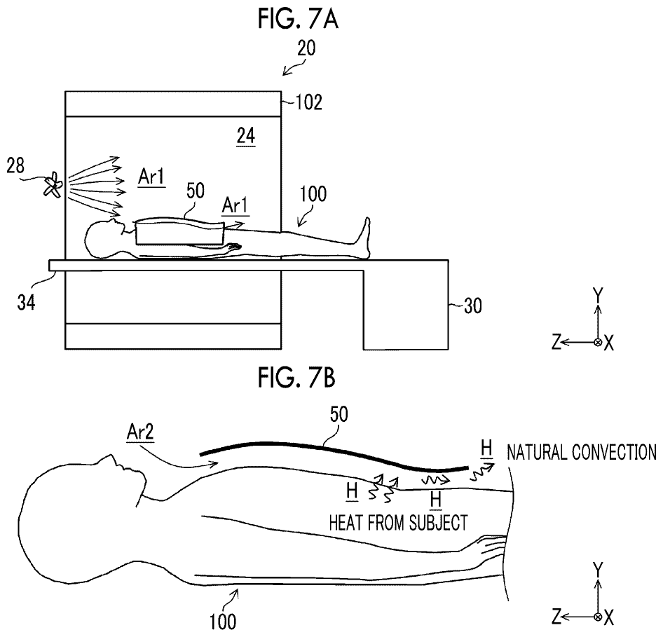

FIGS. 7A and 7B are diagrams for describing a usage form of the receive coil unit 50. FIG. 7A shows a case in which a fan 28 of the MRI apparatus 20 is used, and FIG. 7B shows heat convection using heat from the subject 100.

As shown in FIG. 7A, the fan 28 may be disposed in the vicinity of the imaging space 24 of the static magnetic field generating magnet 102. The fan 28 is installed, for example, on a side opposite to a side on which the table 32 is carried in. The fan 28 blows air Ar1 toward an opening on a carry-in side in order to keep a thermal environment in the imaging space 24 constant. A part of the air Ar1 blown by the fan 28 passes through the first passage 64 (not shown) between the receive coil unit 50 and the subject 100. The heat can be released from between the receive coil unit 50 and the subject 100 to the opening side by the flow of the air Ar1. Since the first passage 64 is formed over the entire region in the Z axis direction, the air Ar1 can pass through the first passage 64 without being obstructed. Although the first passage 64 has been described, the second passage 66 can also allow the air Ar1 to pass through and release heat.

As shown in FIG. 7B, the receive coil unit 50 is used as an abdominal receive coil. In a case in which heat His generated from the subject 100, the heat H tries to be released from the receive coil unit 50, and natural convection occurs. The first passage 64 formed in the receive coil unit 50 promotes the release of the heat H from the subject 100. As shown in FIG. 7B, in a case in which the heat H is released to the outside of the receive coil unit 50, the first passage 64 has a negative pressure with respect to the outside, so that outside air Ar2 flows into the first passage 64 from a side opposite to a direction in which the heat H is released, and thus a flow of air is generated in the first passage 64. The flow of air can promote the release of the heat H. Although the first passage 64 has been described, the heat H can be released through the second passage 66. In addition, the outside air Ar2 flows into the second passage 66, so that the heat H can be more effectively released.

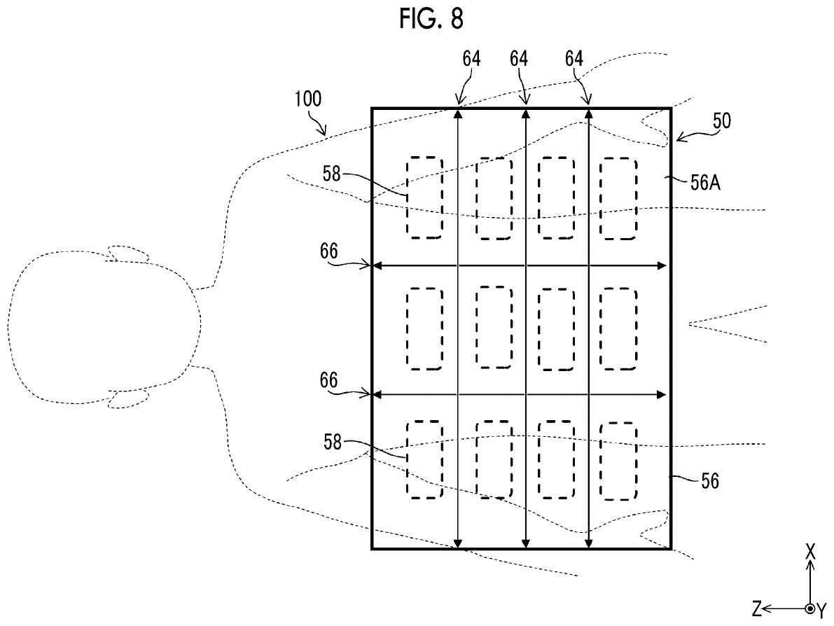

FIG. 8 is a diagram showing a state in which the receive coil unit 50 is mounted on the subject 100 in a direction different from that in FIG. 5. FIG. 8 shows a state in which the receive coil unit 50 of FIG. 5 is rotated clockwise (or counterclockwise) by about 90° as seen from the first sheet body 56A (noncontact outer side surface). In the receive coil unit 50, the first passage 64 and the second passage 66 are formed. Therefore, even in a case in which a mounting orientation of the receive coil unit 50 is changed, heat can be released through the first passage 64 and the second passage 66.

### Structural Example of Passage Forming Member

FIG. 6 illustrates the passage forming member 58 that consists of a single material (one type of material) and that has a plate-like shape. The material and structure of the passage forming member 58 are not limited to the passage forming member 58 shown in FIG. 6 as long as the first passage 64 and the second passage 66 can be formed. Hereinafter, other structures of the passage forming member 58 will be described.

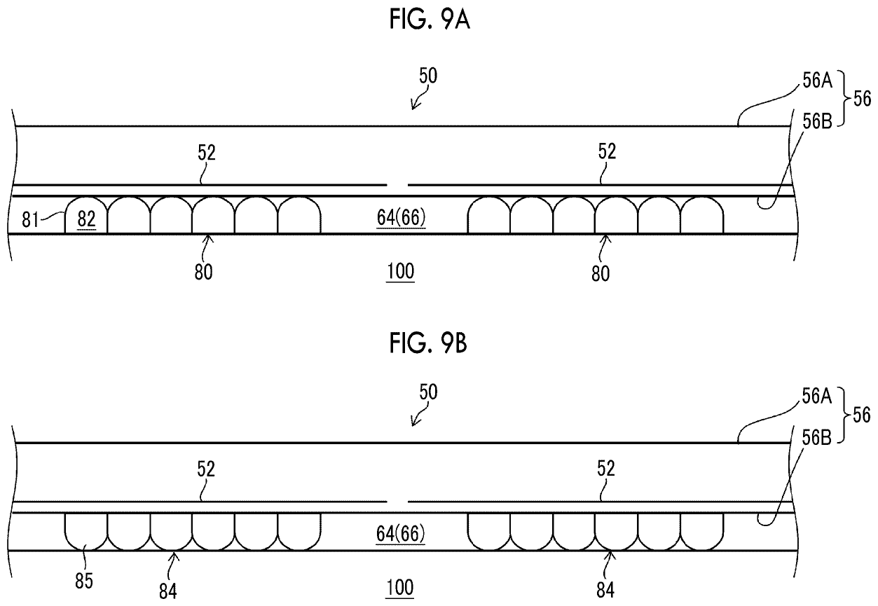

FIGS. 9A and 9B show a case in which passage forming members 80 and 84 are formed of an aggregate in which a plurality of members are aggregated. In a cross-sectional view of

FIG. 9A, the passage forming member 80 is formed of an aggregate in which air bags 81 (independent air bags) are arranged along the coil cover 56. The passage forming member 80 has a plurality of dome-shaped air bags 81 in which air bubbles 82 are sealed, and the passage forming member 80 is formed of an aggregate in which a plurality of the air bags 81 are aggregated. The passage forming members 80 form the first passage 64 and the second passage 66, and each of the passage forming members 80 also functions as a buffer member since it is formed of the air bags 81. Since the passage forming member 80 is an aggregate of the air bags 81, the passage forming member 80 can have deformability. Therefore, in a case in which the receive coil unit 50 is deformed to fit the physique of the subject 100, the passage forming member 80 can also be deformed to fit the physique of the subject 100. Each of the plurality of air bags 81 of the passage forming member 80 basically has the same size and the same shape.

A cross-sectional view of FIG. 9B shows a case in which the passage forming member 84 has a bellows structure. The passage forming member 84 includes a plurality of rod-like members 85 having a solid structure, and the passage forming member 84 is formed of an aggregate in which the rod-like members 85 are arranged along the coil cover 56 to form a bellows structure. The rod-like member 85 is formed of a resin (plastic or the like). Since the passage forming member 84 has a bellows structure, it can be easily deformed between the adjacent rod-like members 85. Therefore, the passage forming members 84 can form the first passage 64 and the second passage 66 and can have deformability. Each of the plurality of rod-like members 85 has basically the same size and the same shape.

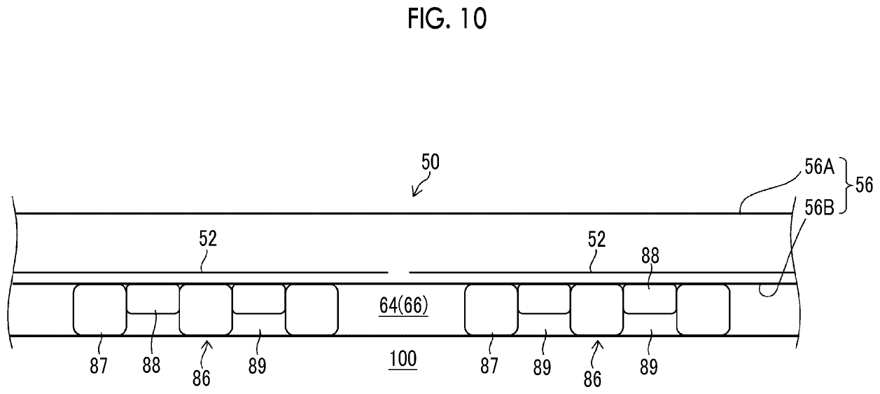

A cross-sectional view of FIG. 10 shows a case in which a passage forming member 86 is formed of an aggregate in which a plurality of members are aggregated and each member constituting the aggregate has a different size and shape. The passage forming member 86 is formed of an aggregate in which a first member 87 and a second member 88 having a size different from the first member 87 are arranged along the coil cover 56. The first member 87 comes into contact with the subject 100, and the second member 88 does not come into contact with the subject 100. The passage forming members 86 forms the first passage 64 and the second passage 66. Further, a groove 89 is formed in the passage forming member 86 due to a difference in thickness between the first member 87 and the second member 88. The groove 89 serves as a passage for air. With this configuration, heat can be released not only from the first passage 64 and the second passage 66 but also from the groove 89 in a region where the passage forming member 86 is disposed. The first member 87 and the second member 88 may be formed of, for example, closed-cell polyurethane.

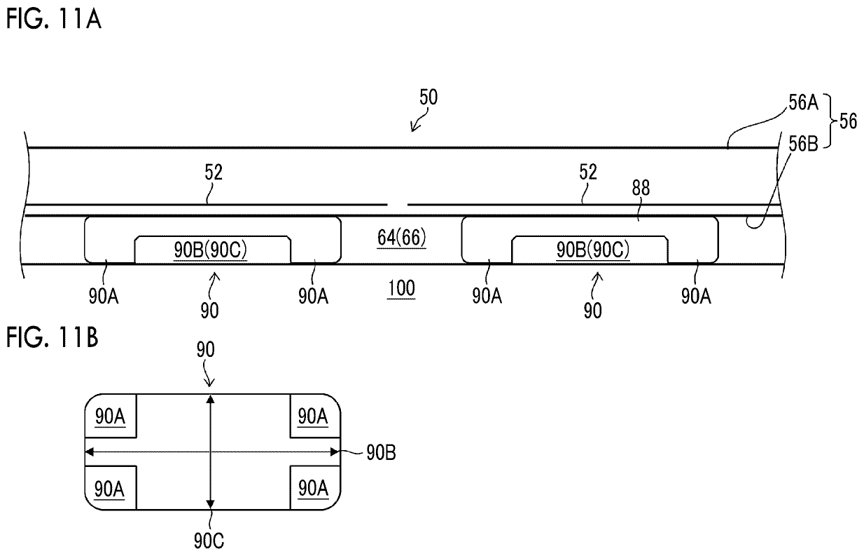

FIG. 11A is a cross-sectional view, and FIG. 11B is a view of a passage forming member 90 as seen from the subject 100 side. A case in which the passage forming member 90 is formed of a single member and a plurality of grooves are formed on a surface of the passage forming member 90 facing the subject 100 is shown. The passage forming member 90 has a plate-like shape as a whole. Four leg portions 90A are provided on the surface of the passage forming member 90 facing the subject 100. The four leg portions 90A form a first groove 90B and a second groove 90C intersecting the first groove 90B in the passage forming member 90. The first groove 90B and the second groove 90C serve as a passage for air. The first groove 90B extends over the entire surface of the passage forming member 90 in the first direction, and the second groove 90C extends over the entire surface of the passage forming member 90 in the second direction different from the first direction. With this configuration, heat can be released not only from the first passage 64 and the second passage 66 but also from the first groove 90B the second groove 90C in a region where the passage forming member 90 is disposed. The passage forming member 90 may be formed of, for example, rubber. The groove 89, the first groove 90B, and the second groove 90C are examples of a groove according to the embodiment of the present invention.

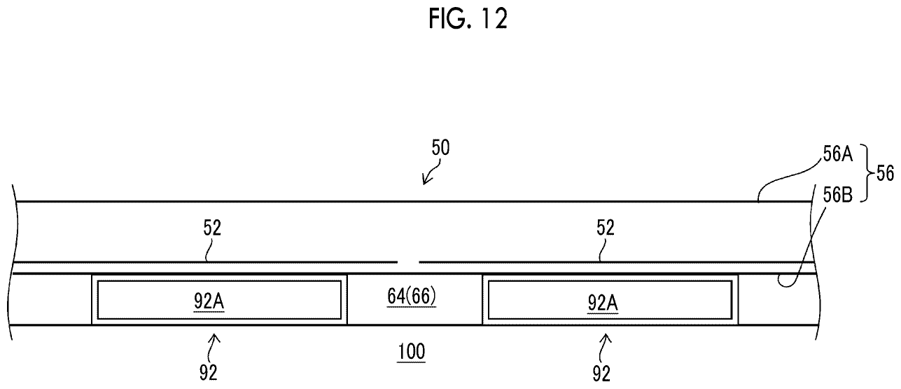

A cross-sectional view of FIG. 12 shows a case in which a passage forming member 92 has a hollow structure. The passage forming member 92 has a tunnel-shaped through-passage 92A that penetrates the passage forming member 92. The through-passage 92A has openings at both ends and extends linearly in one direction. The through-passage 92A serves as a passage for air. With this configuration, heat can be released not only from the first passage 64 and the second passage 66 but also from the through-passage 92A in a region where the passage forming member 90 is disposed. It is preferable to align the direction of the through-passage 92A, since this promotes a flow of air in that direction. The passage forming member 92 may be formed of, for example, a resin or a metal. In addition, through-holes may be formed on both side surfaces of the passage forming member 92. The through-holes on both side surfaces can also serve as a passage for air in a direction intersecting the through-passage 92A.

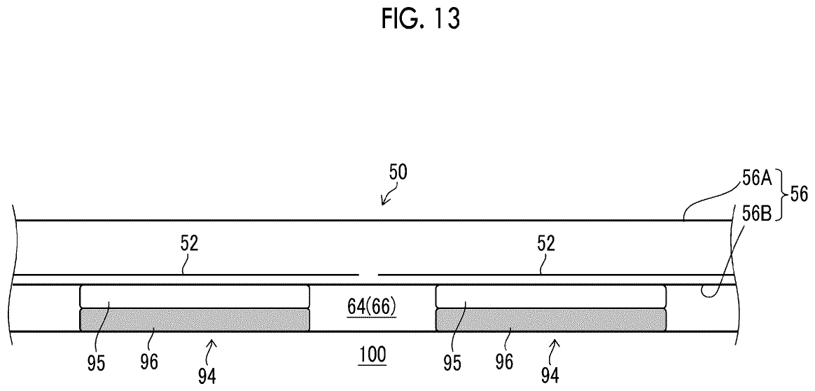

A cross-sectional view of FIG. 13 shows a case in which a passage forming member 94 has a multilayer structure in a thickness direction. The passage forming member 94 has a two-layer structure in which a first member 95 and a second member 96 are laminated in this order from the second sheet body 56B side. Both the first member 95 and the second member 96 have a plate-like shape. In addition, in this example, the first member 95 and the second member 96 have substantially the same thickness and the same shape. On the other hand, materials constituting the first member 95 and the second member 96 can be made different from each other. The second member 96 that comes into contact with the subject 100 can be formed of a material having high cushioning properties (for example, sponge or beads), and the first member 95 can be formed of a material having high load-bearing properties (for example, resin or metal). That is, the passage forming member 94 is formed of a combination of two or more materials. Since the first member 95 mainly defines the shapes of the first passage 64 and the second passage 66 and the second member 96 has cushioning properties, the second member 96 can relieve the contact (roughness or the like) felt by the subject 100 with respect to the passage forming member 94. Although a case in which the passage forming member 94 has a two-layer structure has been exemplified, the passage forming member 94 may have a multilayer structure of three or more layers. In the passage forming member 94, heat can also be released through the first passage 64 and the second passage 66.

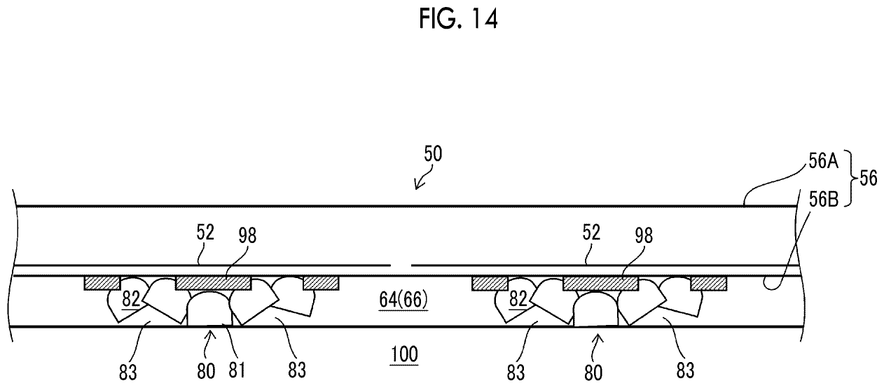

A cross-sectional view of FIG. 14 shows a case in which a spacer 98 is disposed between the passage forming member 80 and the coil cover 56 of the receive coil unit 50. The spacer 98 is provided on the second sheet body 56B (sample contact surface) of the coil cover 56 in a region where the passage forming member 80 is disposed. The spacer 98 is not a member having a uniform thickness, but is formed of members having different thicknesses and has an uneven shape. The passage forming member 80 is disposed to be in contact with the spacer 98. As shown in FIG. 14, the passage forming member 80 has flexibility, so that the passage forming member 80 is deformed to follow the shape of the spacer 98. Therefore, a surface of the passage forming member 80 opposite to a surface that comes into contact with the spacer 98 (a surface of the passage forming member 80 facing the subject 100) has an uneven shape. Due to the uneven shape, a space 83 in which the passage forming member 80 does not come into contact with the subject 100 is formed between the subject 100 and the surface of the passage forming member 80 facing the subject 100. The space 83 serves as a passage for air. With this configuration, heat can be released not only from the first passage 64 and the second passage 66 but also from the space 83 in a region where the passage forming member 80 is disposed. The spacer 98 may be formed of a resin, rubber, a metal, or the like. The spacer 98 is an example of a spacer according to the embodiment of the present invention.

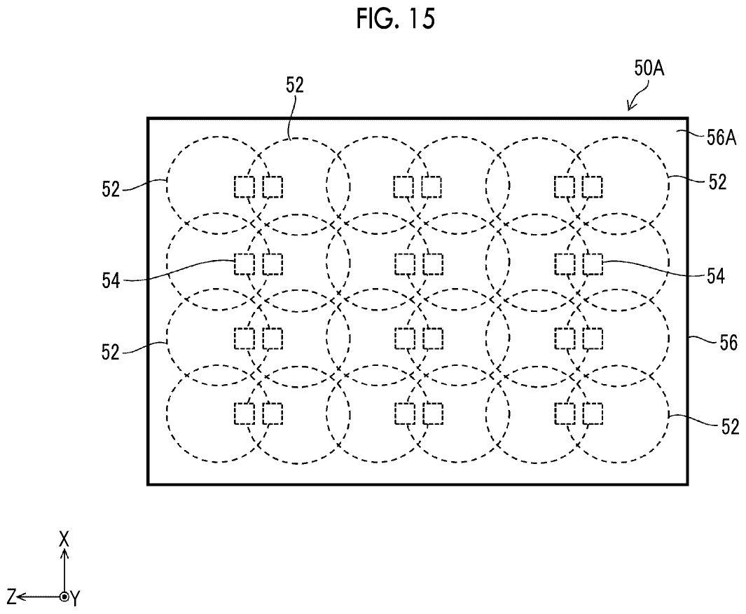

FIG. 15 is a plan view schematically showing another example of the arrangement form of the signal processing circuits 54 that are heat generation sources, and is a view of a receive coil unit 50A as seen from the first sheet body 56A (noncontact outer side surface). The plurality of signal processing circuits 54 are disposed in pairs close to each other in the Z axis direction. In a case of viewing two signal processing circuits 54 as one set in the Z axis direction, a set of three signal processing circuits 54 is disposed. In a case of viewing two signal processing circuits 54 as one set in the X axis direction, a set of four signal processing circuits 54 is disposed.

Next, the preferred arrangement of the passage forming members 58 with respect to the receive coil unit 50A shown in FIG. 15 will be described.

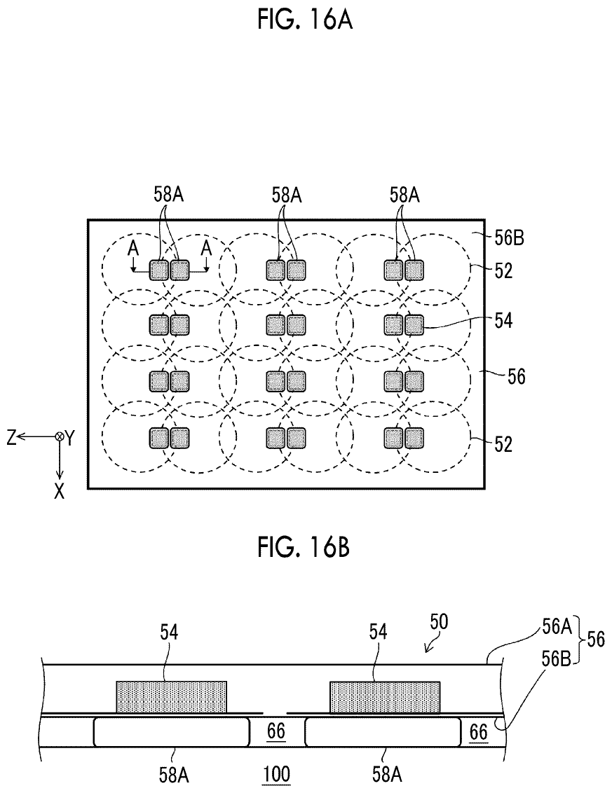

FIG. 16A is a view of the arrangement of passage forming members 58A as seen from the second sheet body 56B (contact outer side surface) of the receive coil unit 50A. FIG. 16B is a view taken along an arrow A of FIG. 16A. As shown in FIGS. 16A and 16B, one passage forming member 58A is disposed at a position corresponding to one signal processing circuit 54. The passage forming member 58A and the signal processing circuit 54 correspond to each other on a one-to-one basis. A second passage 66 extending along the X axis direction is formed between the passage forming members 58A adjacent to each other. Since the passage forming member 58A is disposed at the position corresponding to the signal processing circuit 54, it is possible to prevent the heat generated from the signal processing circuit 54 from being transmitted to the subject 100.

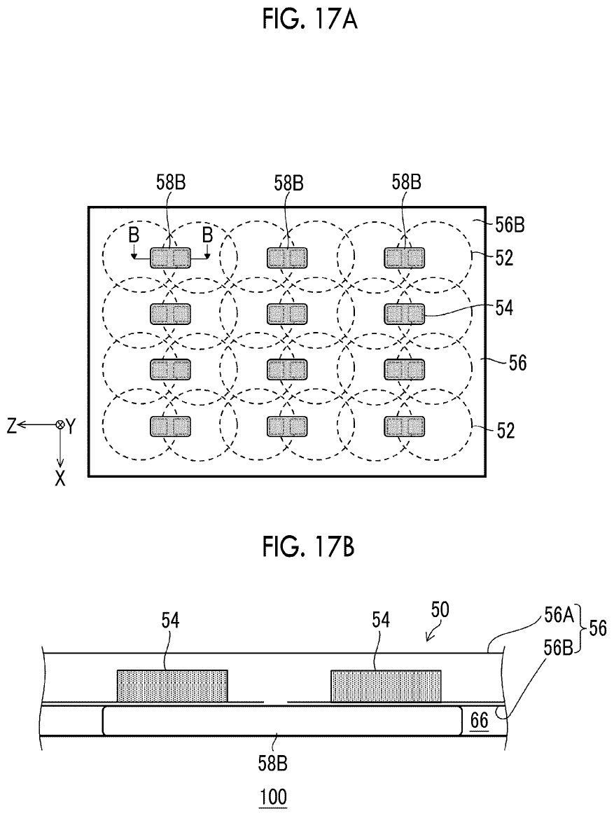

FIG. 17A is a view of the arrangement of passage forming members 58B as seen from the second sheet body 56B (contact outer side surface) of the receive coil unit 50A. FIG. 17B is a view taken along an arrow B of FIG. 17A. As shown in FIGS. 17A and 17B, one passage forming member 58B is disposed at a position corresponding to two signal processing circuits 54. The passage forming member 58B and the signal processing circuit 54 correspond to each other on a one-to-two basis. Since the passage forming member 58B is disposed at the position corresponding to the signal processing circuit 54, it is possible to prevent the heat generated from the signal processing circuit 54 from being transmitted to the subject 100.

The receive coil unit 50A in FIGS. 17A and 17B is advantageous in terms of cost as compared with the receive coil unit 50A of FIGS. 16A and 16B, since the number of the passage forming members 58B can be smaller than the number of the passage forming members 58A.

The passage forming members 58A and 58B have a plate-like shape, but the structure of the other passage forming members described above can be applied. The passage forming members 58A and 58B may be formed of a material having a low thermal conductivity, for example, a resin such as a heat insulating member. As a resin suitable for the heat insulating member, for example, closed-cell polyurethane can be applied.

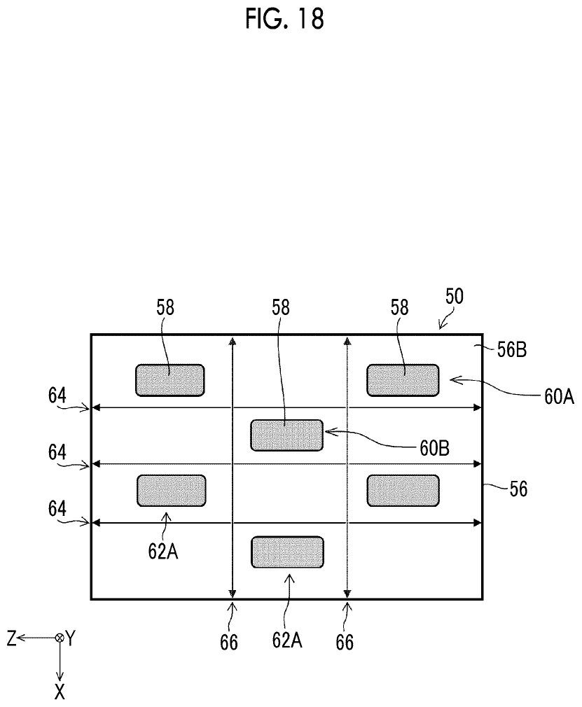

FIG. 18 is a diagram showing arrangement of the passage forming members 58 different from a lattice form in the receive coil unit 50. FIG. 18 is a view of the receive coil unit 50 as seen from the second sheet body 56B (contact outer side surface). The second sheet body 56B has a first direction arrangement group 60A in which two passage forming members 58 are arranged in the Z axis direction, a first direction arrangement group 60B in which one passage forming member 58 is arranged, and a second direction arrangement group 62A in which two passage forming members 58 are arranged in the X axis direction. The passage forming members 58 are arranged in a staggered (zigzag) manner.

The first passage 64 is formed by the first direction arrangement group 60A and the first direction arrangement group 60B adjacent to each other, and the second passage 66 is formed by the second direction arrangement groups 62A adjacent to each other. In the receive coil unit 50 shown in FIG. 18, heat can also be released through the first passage 64 and the second passage 66. In particular, even in a case in which the orientation in which the receive coil unit 50 is mounted on the subject 100 is changed, heat can be released.

### Effects of Embodiment

With the receive coil unit according to the above-described embodiment, the following effects can be obtained. That is, since the first passage and the second passage are formed by the plurality of passage forming members provided in the coil cover, heat can be released from between the subject and the receive coil unit to the outside.

In addition, by forming a groove in the region where the passage forming members is disposed, in addition to the first passage and the second passage, heat can be more effectively released to the outside. In addition, by providing a space in the passage forming member, heat can be more effectively released to the outside.

In the present embodiment, the signal processing circuit 54 including the magnetic coupling prevention circuit is illustrated as the heat generation source of the receive coil unit 50, but the present invention is not limited thereto. For example, the heat generation source may be a magnetic coupling prevention circuit alone that is inserted into the coil element, or a preamplifier that amplifies a detected signal. By disposing the passage forming member 58 at a position corresponding to the heat generation source, it is possible to prevent generated heat from being transmitted to the subject 100.

In the present embodiment, the coil cover 56 of the receive coil unit 50 has a removable bag shape, but the present invention is not limited thereto. For example, it may be embedded with urethane or the like. It is possible to reduce the manufacturing cost by eliminating sewing work and the like.

Further, it is needless to say that the present invention is not limited to the embodiment described above, and that various modifications are possible.

### EXPLANATION OF REFERENCES

10: medical image diagnostic system
50, 50A: receive coil unit
52: coil element
54: signal processing circuit
56: coil cover
58, 58A, 58B, 80, 84, 86, 90, 92, 94: passage forming member
64: first passage
66: second passage
82: air bubble
89: groove
98: spacer
100: subject

What is claimed is:

1. A receive coil unit comprising:
a coil element that receives a nuclear magnetic resonance signal of a subject;
a flexible coil cover that covers a periphery of the coil element; and
a plurality of passage forming members disposed on an outer side surface of the coil cover that comes into contact with the subject, the plurality of passage forming members forming a first passage extending in a first direction and a second passage extending in a second direction different from the first direction between the subject and the coil cover.

2. The receive coil unit according to claim 1,
wherein the passage forming member is formed of one material or a combination of two or more materials selected from the group consisting of a non-magnetic metal, a resin, a closed-cell material, and rubber.

3. The receive coil unit according to claim 1,
wherein the passage forming member has a plate-like shape.

4. The receive coil unit according to claim 1,
wherein the passage forming member is configured to be deformable.

5. The receive coil unit according to claim 1,
wherein the passage forming member is formed of two or more types of members having different shapes.

6. The receive coil unit according to claim 1,
wherein the passage forming member has a shape in which a groove is formed on a subject side.

7. The receive coil unit according to claim 1,
wherein the passage forming member has a hollow structure.

8. The receive coil unit according to claim 1, further comprising:
a heat generation source connected to the coil element,
wherein the passage forming member is disposed at a position corresponding to the heat generation source.

9. The receive coil unit according to claim 1, further comprising:
a heat generation source connected to each of a plurality of the coil elements,
wherein the passage forming member is disposed at a position corresponding to a plurality of the heat generation sources.

10. The receive coil unit according to claim 1,
wherein the passage forming member is formed of a heat insulating member.

11. The receive coil unit according to claim 1, further comprising:
a spacer provided between the passage forming member and the outer side surface of the coil cover.

12. The receive coil unit according to claim 1,
wherein the plurality of passage forming members are arranged in a lattice form.

13. The receive coil unit according to claim 1,
wherein the coil cover has a bag shape.

14. A medical image diagnostic system comprising:
the receive coil unit according to claim 1; and
a magnetic resonance imaging apparatus that processes the nuclear magnetic resonance signal received by the receive coil unit.

15. A coil cover that covers a periphery of a coil element receiving a nuclear magnetic resonance signal of a subject, the coil cover comprising:
a plurality of passage forming members disposed on an outer side surface of the coil cover that comes into contact with the subject, the plurality of passage forming members forming a first passage extending in a first direction and a second passage extending in a second direction different from the first direction between the subject and the coil cover.
